(19) United States
(12) Patent Application Publication
(10) Pub. No.: US 2008/0269742 A1
(43) Pub. Date: Oct. 30, 2008

(54) CONNECTOR ASSEMBLY FOR BONE ANCHORING ELEMENT

(76) Inventors: Mark M. Levy, Raanana (IL); Eyal Zylberberg, Kfar Yona (IL); Yair Spanier, Pardes Hanna (IL)

Correspondence Address: David Klein;DEKEL PATENT LTD., Beit HaRof'im, 18 Menuha VeNahala Street, Room 27, REHOVOT, IL

(21) Appl. No.: 11/739,788

(22) Filed: Apr. 25, 2007

(51) Int. Cl. A61B 17/68

(52) U.S. Cl. 606/60

(57) ABSTRACT

A connector assembly including a bone anchoring element, an articulating interface element including a bone-anchoring-element interface portion extending from a connecting-element interface portion, the bone-anchoring-element interface portion including a plurality of resilient fingers adapted to resiliently clamp on the bone anchoring element, and wherein the connecting-element interface portion includes a pair of arms that define a channel for receiving therein a connector element, and a cap member that fastens on to the arms, wherein tightening of the cap member against the arms causes squeezing of the fingers to clamp them on the bone anchoring element.

### FIELD OF THE INVENTION

The present invention relates generally to anchoring structures for orthopedic devices, and particularly to a connector assembly for connecting rods and the like to bone anchoring elements, such as pedicle screws.

### BACKGROUND OF THE INVENTION

Many spinal fixation systems are found in the prior art for fixing connecting rods and the like to pedicle screws. Some systems have a disadvantage in that the rod must be bent after coupling to the pedicle screw because of anatomical considerations. Some systems attempt to solve this problem with different kinds of connectors that secure the rod to a polyaxial pedicle screw head and permit spatial adjustments before locking the rod in place. It is imperative to lock the connecting rod with respect to the pedicle screw (or any other kind of bone anchoring element) in order to prevent loosening of the spinal fixation system that could have undesirable consequences to the patient. Nevertheless, no design is free of problems and there is still a need for a connector that is “user-friendly” and permits adjustments before locking the rod in place.

### SUMMARY OF THE INVENTION

The present invention seeks to provide an improved connector for connecting rods and the like to bone anchoring elements, such as pedicle screws, as is described more in detail hereinbelow. The connector of the invention permits rotational and translational adjustments before locking the rod in place.

There is thus provided in accordance with an embodiment of the present invention a connector assembly including a bone anchoring element, an articulating interface element including a bone-anchoring-element interface portion extending from a connecting-element interface portion, the bone-anchoring-element interface portion including a plurality of resilient fingers adapted to resiliently clamp on the bone anchoring element, and wherein the connecting-element interface portion includes a pair of arms that define a channel for receiving therein a connector element, a ring placed over the articulating interface element on the bone-anchoring-element interface portion, and a cap member that fastens on to the arms.

The connector assembly may further include a connector element received in the channel, wherein the cap member is tightened against the connector element to force the connector element against the ring, which causes squeezing of the fingers to clamp them on the bone anchoring element. In this embodiment, there is no contact between the connector element and the spherical head of the bone anchoring element.

The resilient fingers may be spaced around a longitudinal axis of the articulating interface element. The resilient fingers may have a wedge portion that flares conically outwards. The arms may define a U-shaped channel. The upper portion of the arms may be threaded, and the cap member may threadedly fasten on to the upper portion of the arms.

### BRIEF DESCRIPTION OF THE DRAWINGS

The present invention will be understood and appreciated more fully from the following detailed description taken in conjunction with the drawings in which:

FIGS. 1A and 1B are simplified pictorial and sectional illustrations, respectively, of a connector assembly for a bone anchoring element, constructed and operative in accordance with an embodiment of the present invention;

FIG. 1C is a more detailed sectional illustration of the connector assembly of FIG. 1B;

FIGS. 2A, 2B, 2C and 2D are simplified pictorial, side-view, front view and side-sectional illustrations, respectively, of an articulating interface element of the connector assembly of FIGS. 1A-1C, constructed and operative in accordance with an embodiment of the present invention;

FIGS. 3A, 3B and 3C are simplified upper pictorial, lower pictorial and side-sectional illustrations, respectively, of a cap member of the connector assembly of FIGS. 1A-1C, constructed and operative in accordance with an embodiment of the present invention;

FIGS. 4A, 4B and 4C are simplified side sectional, upper pictorial and side pictorial illustrations, respectively, of a connector assembly for a bone anchoring element, constructed and operative in accordance with another embodiment of the present invention;

FIGS. 5A, 5B and 5C are simplified side sectional, upper pictorial and side pictorial illustrations, respectively, of a connector assembly for a bone anchoring element, constructed and operative in accordance with yet another embodiment of the present invention;

FIGS. 6A, 6B and 6C are simplified side sectional, upper pictorial and side pictorial illustrations, respectively, of a connector assembly for a bone anchoring element, constructed and operative in accordance with still another embodiment of the present invention;

FIGS. 7A, 7B and 7C are simplified side sectional, upper pictorial and side pictorial illustrations, respectively, of a connector assembly for a bone anchoring element, constructed and operative in accordance with another embodiment of the present invention, comprising an internal finger ring;

FIGS. 8A, 8B and 8C are simplified front, side and upper pictorial illustrations, respectively, of a connector assembly for a bone anchoring element, constructed and operative in accordance with another embodiment of the present invention, comprising two tightening elements that tighten in opposite directions; and

FIGS. 9A and 9B are simplified side sectional illustrations of a connector assembly for a bone anchoring element, constructed and operative in accordance with another embodiment of the present invention, which tightens with wedging action.

### DETAILED DESCRIPTION OF EMBODIMENTS

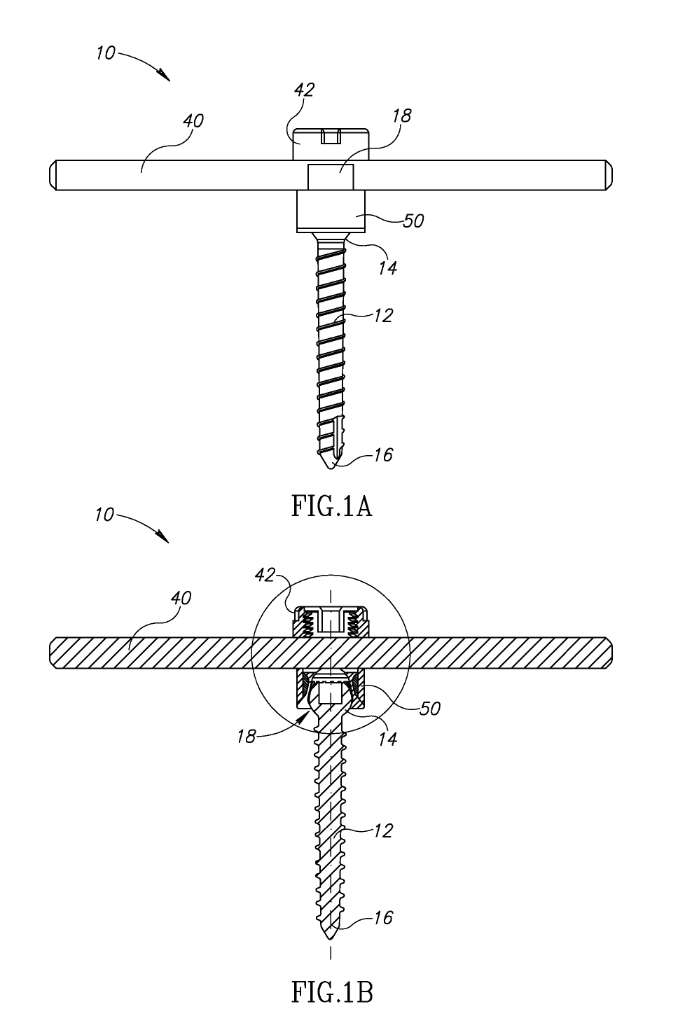

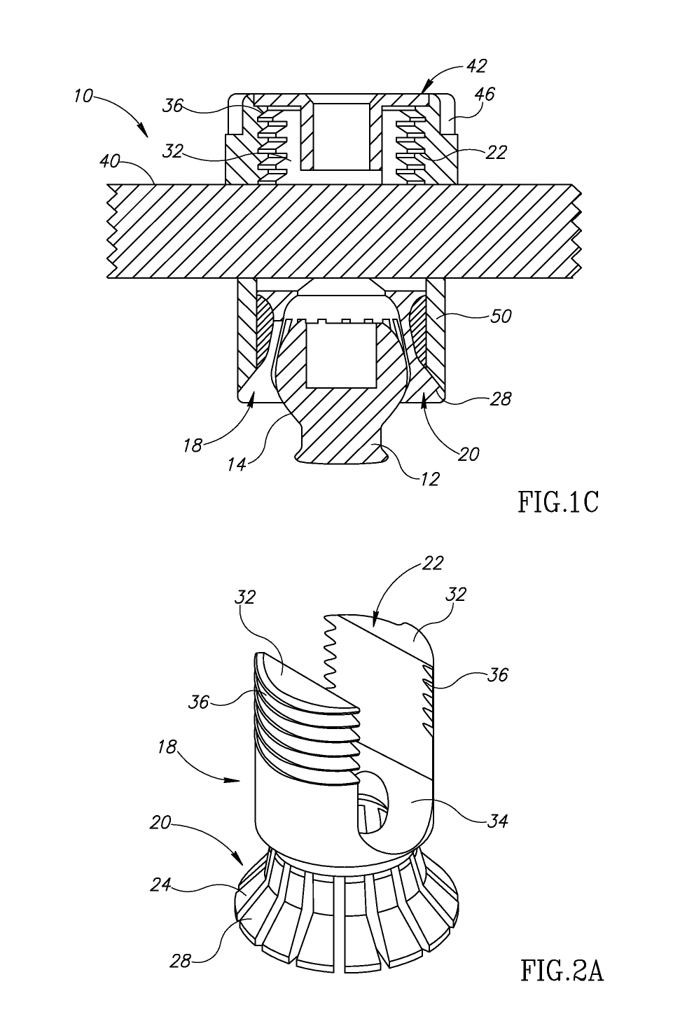

Reference is now made to FIGS. 1A-1C, which illustrate a connector assembly 10 for a bone anchoring element 12, constructed and operative in accordance with an embodiment of the present invention.

In a non-limiting embodiment of the invention, the bone anchoring element 12 includes a threaded mechanical fastener (for example, without limitation, a pedicle screw, a pedicle anchor device, a blocking screw for a pedicle anchoring device or any other device for attachment to the pedicle, vertebral body or any other bone) having a spherical proximal portion 14 (also referred to as a spherical head 14) and a (pointed) distal portion 16. Alternatively, bone anchoring element 12 can be a hook or other mechanical fastener with a spherical head. The spherical head 14 may be formed with a socket for an Allen wrench or the like.

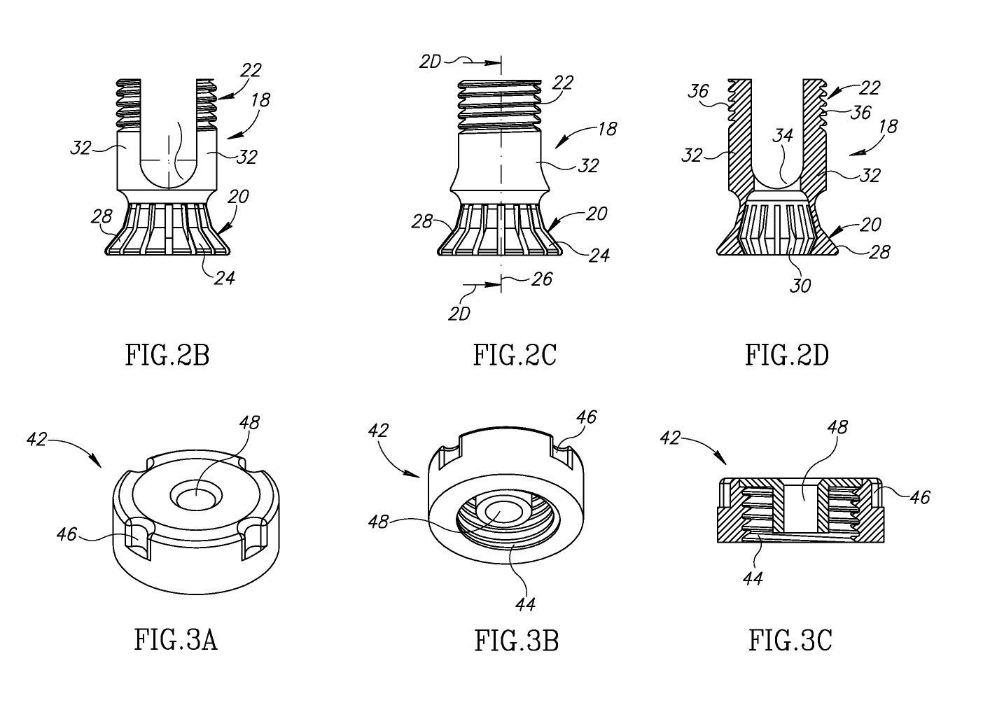

Reference is now made additionally to FIGS. 2A-2D. The connector assembly 10 may include an articulating interface element 18 that includes a bone-anchoring-element interface portion 20 extending from a connecting-element interface portion 22. Bone-anchoring-element interface portion 20 may include a plurality of resilient fingers 24 (also referred to as tabs, leaves or petals, these terms and the like being used interchangeably) spaced around a longitudinal axis 26 thereof. Resilient fingers 24 may have a wedge portion 28 that flares conically outwards. The inside surfaces of resilient fingers 24 define a clamping portion 30 (FIG. 2D) that resiliently clamps on spherical head 14. Connecting-element interface portion 22 may include a pair of arms 32 that define a U-shaped channel 34, shaped to receive therein a connector element 40. An upper portion 36 of arms 32 may be threaded.

Reference is now made to FIGS. 3A-3C, which illustrate a cap member 42 of the connector assembly 10. Cap member 42 may include an internally threaded portion 44 that screws on to upper portion 36 of arms 32. Cap member 42 may be formed with tightening tool (e.g., modified type of screwdriver) interfaces 46, such as indentations formed on the periphery of cap member 42 that permit grasping and turning cap member 42 with a tightening tool to tighten it on to upper portion 36 of arms 32. Cap member 42 may also be formed with a central hub 48 that may have a centering hole for centering the tightening tool. This is of course just one non-limiting example of structure for turning and tightening cap member 42, and many other possibilities exist within the scope of the invention. This is just one non-limiting example of structure for the cap member 42, and other structures may be used to carry out the invention, such as but not limited to, an internally threaded set screw, with or without a washer.

A ring 50 (FIGS. 1A-1C) may be provided for placing on bone-anchoring-element interface portion 20.

The connector assembly 10 may be constructed of any suitable, medically safe material, such as but not limited to, stainless steel alloy (e.g., AISI 316L), titanium or titanium alloy or chrome cobalt alloy, PEEK, shape memory alloys or polymers, including resorbable materials, or any combination of the above. Any material may be coated, such as with HA, any factors or substances including active or passive antibiotics, etc. Different parts of connector assembly 10 may be constructed of different materials with different resilience or hardness, or of the same material but treated to have different resilience or hardness.

The connector element 40 is illustrated as a rod with a cylindrical cross section. However, the invention is not limited to this construction, and connector element 40 may be any slender elongate element, such as but not limited to, a bar of hexagonal, rectangular or square cross section, a rod of elliptical cross section, and many others.

Assembly of connector assembly 10 is straightforward and easy. After bone anchoring element 12 (e.g., pedicle screw) is in place, the user places the bone-anchoring-element interface portion 20 of articulating interface element 18 over spherical head 14 of bone anchoring element 12. Ring 50 is then placed over articulating interface element 18 and sits against wedge portion 28. The connector element 40 is placed in U-shaped channel 34 of connecting-element interface portion 22. Cap member 42 is then tightened to tighten central hub 48 against connector element 40. This forces connector element 40 against ring 50, which presses wedge portion 28 and squeezes fingers 24 to clamp them on spherical head 14 of bone anchoring element 12. There is no contact between connector element 40 and spherical head 14 of bone anchoring element 12.

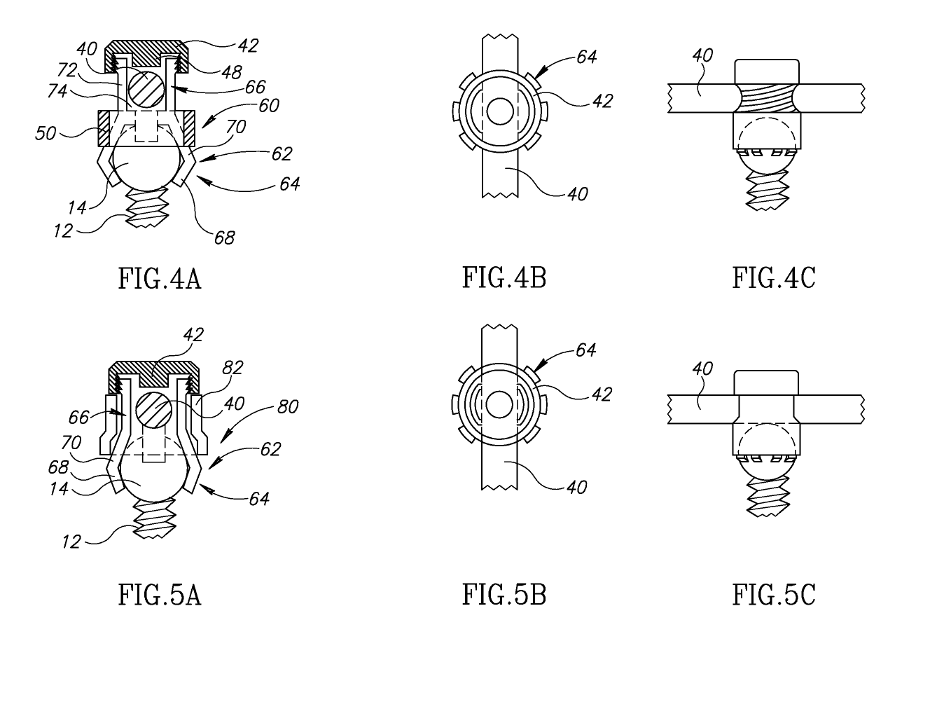

Reference is now made to FIGS. 4A, 4B and 4C, which illustrate a connector assembly 60 for a bone anchoring element, constructed and operative in accordance with another embodiment of the present invention.

The connector assembly 60 may include an articulating interface element 62 that includes a bone-anchoring-element interface portion 64 extending from a connecting-element interface portion 66. Bone-anchoring-element interface portion 64 may include a plurality of resilient fingers 68 spaced around a longitudinal axis thereof. Resilient fingers 68 may have a wedge portion 70 that flares conically outwards. The inside surfaces of resilient fingers 68 define a clamping portion that resiliently clamps on spherical head 14 of bone anchoring element 12. Connecting-element interface portion 66 may include a pair of arms 72 that define a channel 74, shaped to receive therein connector element 40. The upper portion of arms 72 is threaded to receive cap member 42.

Assembly of connector assembly 60 is straightforward and easy. The user places the bone-anchoring-element interface portion 64 over spherical head 14 of bone anchoring element 12. Ring 50 is then placed over articulating interface element 62 and sits against wedge portion 70. The connector element 40 is placed in channel 74 of connecting-element interface portion 66. Cap member 42 is then tightened to tighten central hub 48 against connector element 40. This forces connector element 40 against ring 50, which presses wedge portion 70 and squeezes fingers 68 to clamp them on spherical head 14 of bone anchoring element 12. There is no contact between connector element 40 and spherical head 14 of bone anchoring element 12.

The embodiment of FIGS. 4A-4C is similar to the embodiment of FIGS. 3A-3C. One of the differences is in the position of the wedge portion. In the embodiment of FIGS. 3A-3C, the wedge portion is at the lower hemisphere of spherical head 14. In the embodiment of FIGS. 4A-4C, the wedge portion is at the upper hemisphere of spherical head 14.

Reference is now made to FIGS. 5A, 5B and 5C, which illustrate a connector assembly 80 for a bone anchoring element, constructed and operative in accordance with yet another embodiment of the present invention. Connector assembly 80 is similar to connector assembly 60, with like elements being designated by like numerals.

Connector assembly 80 differs from connector assembly 60 in that connector assembly 80 employs a ring 82 that not only contacts wedge portion 70 but also extends upwards for abutting against the bottom edge of cap member 42. When cap member 42 is tightened to tighten central hub 48 against connector element 40, cap member 42 also pushes down against ring 82, which presses wedge portion 70 and squeezes fingers 68 to clamp them on spherical head 14 of bone anchoring element 12. Thus cap member 42 directly pushes wedge portion 70 and not indirectly through connector element 40. There is contact between connector element 40 and spherical head 14 of bone anchoring element 12.

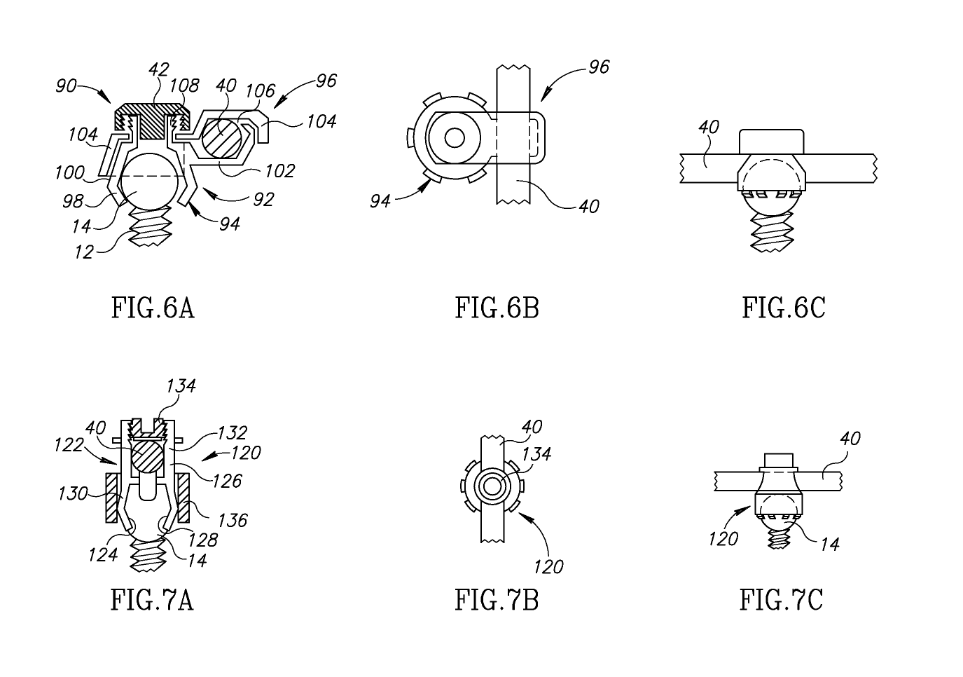

Reference is now made to FIGS. 6A, 6B and 6C, which illustrate a connector assembly 90 for a bone anchoring element, constructed and operative in accordance with still another embodiment of the present invention.

The connector assembly 90 may include an articulating interface element 92 that includes a bone-anchoring-element interface portion 94 extending from a connecting-element interface portion 96. Bone-anchoring-element interface portion 94 may include a plurality of resilient fingers 98 spaced around a longitudinal axis thereof. Resilient fingers 98 may have a wedge portion 100 that flares conically outwards. The inside surfaces of resilient fingers 98 define a clamping portion that resiliently clamps on spherical head 14 of bone anchoring element 12. Connecting-element interface portion 96, unlike the previous embodiments, is offset from the longitudinal axis of bone anchoring element 12. Connecting-element interface portion 96 may include a pair of arms. One arm 102 extends sideways from wedge portion 100 of articulating interface element 92. Another arm 104 has one portion that fits over arm 102 to define a channel 106, shaped to receive therein connector element 40. Another portion of arm 104 sits against wedge portion 100. An upper portion 108 of connecting-element interface portion 96 is threaded to receive cap member 42.

Assembly of connector assembly 90 is straightforward and easy. The user places the bone-anchoring-element interface portion 94 over spherical head 14 of bone anchoring element 12. The connector element 40 is placed in channel 106 on arm 102 of connecting-element interface portion 96. Arm 104 is then placed over connector element 40 and sits against wedge portion 70. Cap member 42 is then tightened to tighten arm 104 against connector element 40. Arm 104 also presses wedge portion 100 and squeezes fingers 98 to clamp them on spherical head 14 of bone anchoring element 12. There is no contact between connector element 40 and spherical head 14 of bone anchoring element 12. It is noted that arm 104 may be hinged to arm 102 or connected with an extra fastener (e.g., screw) for improved locking.

Reference is now made to FIGS. 7A, 7B and 7C, which illustrate a connector assembly 120 for a bone anchoring element, constructed and operative in accordance with another embodiment of the present invention.

The connector assembly 120 may include an articulating interface element which is shaped and also referred to as an internal finger ring 122. Finger ring 122 includes a bone-anchoring-element interface portion 124 extending from a connecting-element interface portion 126. Bone-anchoring-element interface portion 124 may include a plurality of resilient fingers 128 spaced around a longitudinal axis thereof. Resilient fingers 128 may have a wedge portion 130 that flares conically outwards. Fingers 128 define a clamping portion that resiliently clamps on spherical head 14 of bone anchoring element 12. Connecting-element interface portion 126 may include a pair of arms 132 that define a channel, shaped to receive therein connector element 40. The upper portion of arms 132 is threaded to receive cap member 134. A ring 136 is placed around fingers 128.

In the assembly of connector assembly 120, instead of the ring wedging against the wedge portion of the fingers as in the previous embodiments, the opposite is true. By tightening cap member 134 against connector element 40, fingers 128 and wedge portion 130 are forced and wedged outwards against the internal circumference of the rigid ring 136. This internal wedging force tightens fingers 128 and wedge portion 130 against spherical head 14.

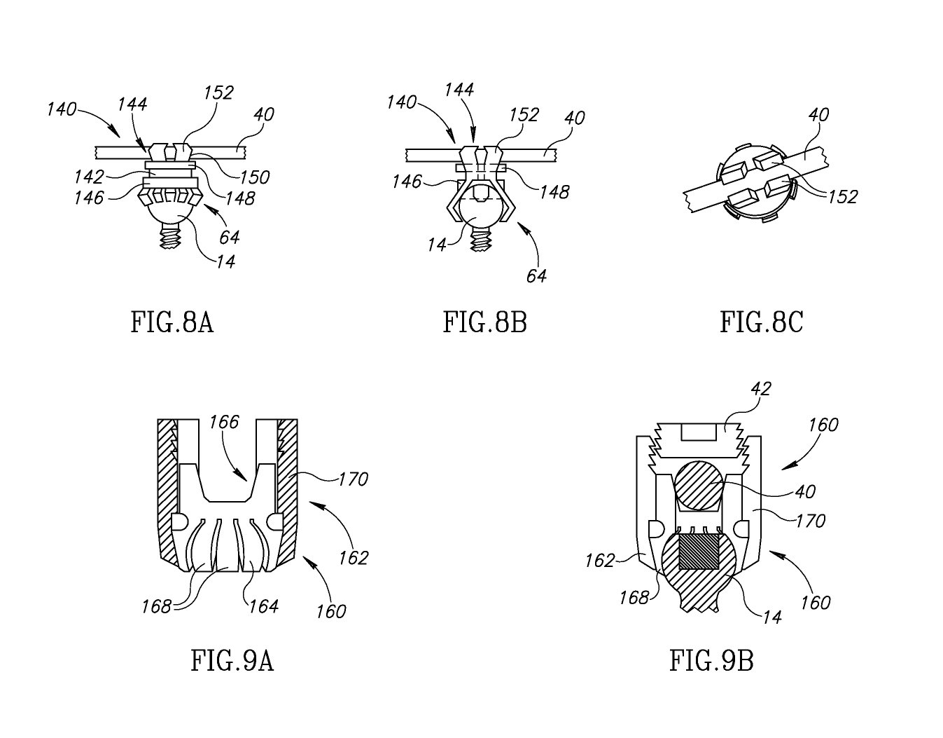

Reference is now made to FIGS. 8A, 8B and 8C, which illustrate a connector assembly 140 for a bone anchoring element, constructed and operative in accordance with another embodiment of the present invention. Connector assembly 140 is constructed similar to connector assembly 60 of FIGS. 4A-4C, with like elements being designated by like numerals. Connector assembly 140 may include a tightening portion 142, which is a threaded shaft that extends between bone-anchoring-element interface portion 64 and connecting-element interface portion 144. A lower (threaded) ring or nut 146 may be tightened on tightening portion 142 against the wedge portion of bone-anchoring-element interface portion 64, as described above. In addition, another (upper) ring or nut 148 (also threaded) may be tightened on tightening portion 142 against a wedge portion 150 of connecting-element interface portion 144, which in turn tightens fingers 152 of connecting-element interface portion 144 on to connector element 40. Thus, the two tightening elements 146 and 148 tighten in opposite directions.

Reference is now made to FIGS. 9A and 9B which illustrate a connector assembly 160 for a bone anchoring element, constructed and operative in accordance with another embodiment of the present invention.

The connector assembly 160 may include an articulating interface element 162 that includes a bone-anchoring-element interface portion 164 extending from a connecting-element interface portion 166. Bone-anchoring-element interface portion 164 may include a plurality of resilient fingers 168 spaced around a longitudinal axis thereof. The articulating interface element 162 is disposed inside a wedging ring 170, which has an upper end that is threaded to receive cap member 42. The action of tightening cap member 42 onto ring 170 causes cap member 42 to press against connector element 40 and force articulating interface element 162 with its fingers 168 to move downwards with respect to wedging ring 170. This action wedges and forces fingers 168 against spherical head 14.

The connector assemblies of the invention permit rotational adjustments of the articulating interface element with respect to the spherical head of the bone anchoring element and translational adjustments of the connector element before locking the connector element in place.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A connector assembly comprising:
a bone anchoring element;
an articulating interface element comprising a bone-anchoring-element interface portion extending from a connecting-element interface portion, said bone-anchoring-element interface portion comprising a plurality of resilient fingers adapted to resiliently clamp on said bone anchoring element, and wherein said connecting-element interface portion comprises a pair of arms that define a channel for receiving therein a connector element; and
a cap member that fastens on to said arms, wherein tightening of said cap member against said arms causes squeezing of said fingers to clamp them on said bone anchoring element.

2. The connector assembly according to claim 1, further comprising a connector element received in said channel and a ring placed over said articulating interface element that contacts a wedge portion formed on said bone-anchoring-element interface portion, wherein said cap member is tightened against said connector element to force said connector element against said ring, which causes squeezing of said fingers to clamp them on said bone anchoring element.

3. The connector assembly according to claim 1, wherein there is no contact between said connector element and said spherical head of said bone anchoring element.

4. The connector assembly according to claim 1, wherein said resilient fingers are spaced around a longitudinal axis of said articulating interface element.

5. The connector assembly according to claim 1, wherein said arms define a U-shaped channel.

6. The connector assembly according to claim 1, wherein an upper portion of said arms is threaded, and said cap member threadedly fastens on to the upper portion of said arms.

7. The connector assembly according to claim 1, further comprising a connector element received in said channel and a ring placed over said articulating interface element that contacts a wedge portion formed on said bone-anchoring-element interface portion, said ring having an extension for abutting against a bottom edge of said cap member, wherein when said cap member is tightened against said connector element, said cap member also applies a force against said ring, which presses said wedge portion which causes squeezing of said fingers to clamp them on said bone anchoring element.

8. The connector assembly according to claim 1, wherein said connecting-element interface portion is offset from a longitudinal axis of said bone anchoring element.

9. The connector assembly according to claim 8, wherein a first arm of said connecting-element extends sideways from a wedge portion of said articulating interface element and a second arm of said connecting-element has one portion that fits over the first arm to define said channel, said second arm having a portion that sits against said wedge portion.

10. The connector assembly according to claim 9, further comprising a connector element received in said channel, and wherein tightening of said cap member tightens said second arm against said connector element, and also causes said second arm to press against said wedge portion and squeeze said fingers to clamp them on said bone anchoring element.

11. The connector assembly according to claim 1, further comprising a connector element received in said channel and a ring placed over said articulating interface element wherein tightening said cap member against said connector element wedges said fingers outwards against an internal circumference of said ring and clamps said fingers on said bone anchoring element.

12. The connector assembly according to claim 1, further comprising a tightening portion that extends between said bone-anchoring-element interface portion and said connecting-element interface portion, wherein two tightening elements are assembled on said tightening portion, one of the tightening elements tightening against said bone-anchoring-element interface portion and the other tightening element tightening against said connecting-element interface portion.

13. The connector assembly according to claim 1, wherein said articulating interface element is disposed inside a wedging ring, said wedging ring having an upper end that receives said cap member, and wherein tightening said cap member onto said wedging ring forces said articulating interface element with said resilient fingers to move with respect to said wedging ring and clamp said fingers on said bone anchoring element.

14. A connector assembly comprising:
a bone anchoring element comprising a spherical head;
an articulating interface element comprising a bone-anchoring-element interface portion extending from a connecting-element interface portion, said bone-anchoring-element interface portion comprising a plurality of resilient fingers spaced around a longitudinal axis thereof, said resilient fingers having a wedge portion that flares conically outwards, wherein inside surfaces of said resilient fingers define a clamping portion that resiliently clamps on said spherical head, and wherein said connecting-element interface portion comprises a pair of arms that define a U-shaped channel, an upper portion of said arms being threaded;
a connector element received in said U-shaped channel;
a ring placed over said articulating interface element on said bone-anchoring-element interface portion against said wedge portion; and
a cap member comprising an internally threaded portion that screws on to the upper portion of said arms, wherein said cap member is tightened against said connector element to force said connector element against said ring, which presses said wedge portion and squeezes said fingers to clamp them on said spherical head of said bone anchoring element.

15. A connector assembly according to claim 14, wherein there is no contact between said connector element and said spherical head of said bone anchoring element.

16. A connector assembly according to claim 14, wherein there is contact between said connector element and said spherical head of said bone anchoring element.
